(19) United States
(12) Patent Application Publication
(10) Pub. No.: US 2017/0166495 A1
(43) Pub. Date: Jun. 15, 2017

(54) PROCESS FOR PRODUCTION OF HYDROCARBONS BY OCM

(71) Applicant: Haldor Topsøe A/S, Kgs. Lyngby (DK)

(72) Inventors: Jens Michael Poulsen, Lyngby (DK); Francois-Xavier Chiron, Copenhagen (DK)

(21) Appl. No.: 15/325,772

(22) PCT Filed: Jul. 17, 2015
(86) PCT No.: PCT/EP2015/066444

§ 371 Date: Jan. 12, 2017

(30) Foreign Application Priority Data
Jul. 22, 2014 (DK) PA 2014 00403

(51) Int. Cl. C07C 2/82; C07C 5/03; C10G 50/00; C07C 1/04

(52) U.S. Cl. CPC C07C 2/82; C07C 1/0485; C07C 5/03; C10G 50/00; C10G 2400/02; C10G 2300/1025; C10G 2400/30

(57) ABSTRACT

In the present application is described a plant and process for production of hydrocarbons, the process comprising the steps of: —converting at least a feed stream comprising CH4 and O2 feed into a conversion effluent by oxidative coupling of methane (OCM), —withdrawing one or more product streams from the conversion effluent by at least one product retrieval stage; —recycling at least a portion of the remaining conversion effluent as a recycle stream in a recycle loop, said recycle loop comprising a hydrogenation stage and a methanation stage; and —adding at least part of the recycle stream to the feed stream.

Methane may be converted into ethylene by the Oxidative Coupling of Methane processes (OCM). The conversion is carried out according to the exothermic reaction (ΔH=−280 kJ/mol) 2CH₄+O₂→C₂H₄+2H₂O at high temperatures in the range 600-950° C.

Generally, the conversion rate in OCM processes is relatively low which leaves a need for methods for optimizing the output and efficiency of the overall OCM setup.

These and other needs are met by a process for production of hydrocarbons, the process comprising the steps of:
converting at least a feed stream comprising CH₄ and an O₂ feed into a conversion effluent comprising Ethylene by oxidative coupling of methane (OCM);
withdrawing one or more product streams from the conversion effluent by at least one product retrieval stage;
recycling at least a portion of the remaining conversion effluent as a recycle stream in a recycle loop, said recycle loop comprising a hydrogenation stage and a methanation stage; and
adding at least part of the recycle stream to the feed stream whereby a higher methane content in the OCM feed is obtained and thus the overall efficiency of the process is increased as the treated, at least hydrogenated and methanated part of the effluent is recycled to allow further conversion of unreacted CH₄, as well as conversion of the formed CO/CO₂/H₂ which is methanated to form more CH₄ according to the process. Furthermore, the composition of the recycle is optimized (e.g. minimizing or lowering H₂ and CO concentrations) to the OCM process by the stages introduced in the recycle loop.

A stage may be a separate reactor and/or one or more sections or layers in a reactor.

Hydrogenation may be defined as the reaction between molecular hydrogen and unsaturated hydrocarbons such as ethylene, acetylene, propylene, methylacetylenyielding to the corresponding alkane.

Hydrogenation of CO and CO₂ may be defined as methanation.

The conversion effluent comprises Ethylene as well as it may comprise unconverted ethane and higher olefins such as propylene, butene and higher alkenes such as propane, butane, cyclic molecules and aromatics.

If methanation is carried out on its own, the unsaturated hydrocarbons e.g. containing carbon double- or triple-bonds will form carbon on the methanation catalyst, thereby deactivating it and building up undesirable pressure drop.

Unsaturated hydrocarbons in problematic levels will exist in effluent both in an “Ethylene case” where Ethylene is extracted directly from the OCM, and in “gasoline/aromatics cases” where the OCM effluent is processed to gasoline or aromatics over a gasoline/aromatics synthesis catalyst.

The combination of hydrogenation of unsaturated HCs and subsequent methanation is critical for the techno-economic feasibility of OCM-based systems.

Contrary to recycle treatment as known from e.g. Fischer-Tropsch where the composition of the recycle is optimized to have a high H₂ content, one of the present requirements for OCM are a recycle gas with low H₂ and CO contents. Thus the present recycle loop is specifically arranged to increase the efficiency of the present OCM conversion and feed gas.

OCM has inherently a relatively low conversion. If no recycle is established, the efficiency will be low and significant value will be lost in large amounts of low-value by-product (methane, syngas, unsaturated hydrocarbons such as olefins, higher hydrocarbons). In order to maximize value, it is here proposed to recycle unconverted methane as well as the majority of valuable byproducts (syngas, unsaturated hydrocarbons such as olefins, higher hydrocarbons). However, the byproduct stream cannot advantageously be recycled as-is. The present process and plant provides an efficient solution with respect to both feed and energy consumption of the overall setup.

An efficient recycle poses a non-trivial challenge solved by the present, both in the case of e.g. Ethylene production, and even more so when the OCM effluent is processed over a gasoline/aromatization catalyst, generating products including unsaturated hydrocarbons such as olefins and higher hydrocarbons.

For example H₂ constitutes an efficiency challenge in the OCM process as reaction with O₂ forms undesired H₂O and lowers O₂-efficiency. Thus, it may be advantageous to ensure that the recycle stream added to the feed stream has a relatively low H₂ content.

CO is undesirable in the OCM process for the same reasons (forming CO2). Saturated hydrocarbons fed to the OCM are valuable since they may be converted to valuable product such as ethylene or propylene.

Unsaturated hydrocarbons in the form of ethylene, acetylene, propylene, methylacetylene or higher h.c. comprising aromatic molecules can represent as high as 1 vol % of the methanator feed, both in the case where the effluent is from an OCM reactor, and where it has been processed through a secondary synthesis for gasoline or aromatics. These unsaturated hydrocarbons are undesirable in the methanation section since they favor carbon formation which leads to methanation catalyst deactivation and pressure drop build up. It is essential for the overall feasibility to hydrogenate these molecules. The use of a commercial Co—Mo or Cu-based or other hydrogenation catalyst will help in saturating these molecules. Furthermore a Cu-based catalyst has also shift activity which can help in limiting the temperature rise in the downstream methanators. Saturated hydrocarbons (for example Ethane) will pass through the methanators without being converted. They will advantageously be fed to the OCM reactor to yield a valuable product.

If the recycle loop further comprises a CO₂ addition stage upstream the methanation stage it is possible to optimize the composition of the recycle stream for an efficient methanation in the methanation stage, maximizing CH₄ generation and minimizing H₂. In the methanation stage H₂, CO, CO₂ is reacted into CH₄ and the preferred module/ratio between reactants in the stream entering the methanation stage is

$${M}_{F} = \frac{{H}_{2} - {CO}_{2} + {O}_{2} + {C}_{2} {H}_{4} + 2 {C}_{2} {H}_{6}}{CO + {CO}_{2} + {O}_{2} + {C}_{2} {H}_{4} + {C}_{2} {H}_{6}} = 3.00$$

i.e. in case C₂H₄=0 and O₂=0;

$$M = \frac{{H}_{2} - {CO}_{2} + 2 {C}_{2} {H}_{6}}{CO + {CO}_{2} + {C}_{2} {H}_{6}} = 3.00$$

Preferably CO₂ is added upstream the methanation stage at the CO₂ addition stage thereby ensuring that near stoichiometric equilibrium conditions or an CO₂ surplus in the methanation step can be obtained. If a methanation stage or the recycle loop comprises more than one reactor CO₂ may alternatively be added before or between the reactors.

H₂O may be at least partially removed at one or more points in recycle loop before and/or after the product retrieval stages. In relation to methanation H₂O may advantageously be removed before one or more of the involved methanation steps in order to move equilibrium in favor of CH₄. However some H₂O may help avoid carbon formation.

H₂O may be removed by simple condensation or by other known means. If needed H₂O may be added e.g. by recycle of a H₂O containing stream. Also steam may be added e.g. upstream the hydrogenation stage.

The product obtained at the one or more product steps may e.g. be ethylene and/or CO₂. Ethylene is produced in the OCM process and may be obtained from the effluent as one of one or more products. CO₂ may be withdrawn from the effluent as CO₂ in most embodiments is a dilutent without value downstream. This and/or otherwise provided CO₂ may in some embodiments be used for addition at the CO₂ addition stage later in the recycle loop to obtain near-ideal stoichiometry or excess CO₂ for methanation.

The ethylene can be extracted as product, with or without by-products in one or more of the product retrieval stages or be processed further.

In various embodiments at least one of the one or more products obtained at the one or more product steps may e.g. aromatics and/or raw gasoline.

By aromatics is understood a stream comprising hydrocarbons with a high content (typically more than 40%) of C6 to C10 aromatic molecules such as Benzene, Toluene, Xylenes, tri-methyl-benzenes and/or tetra-methyl-benzenes.

Where at least part of the OCM effluent is converted into aromatics and/or raw gasoline it may be advantageous if CO₂ is withdrawn from the OCM effluent upstream the conversion to aromatics/gasoline in order to optimize the reaction conditions in the aromatics/gasoline synthesis.

The tail gas from the aromatics and/or raw gasoline synthesis may comprise a mixture of CH₄, CO, CO₂, C₂H₄, C₂H₆ wherein CH₄ is found in a concentration of around (molar %) 80%-90%, H₂ 5-10%, and/or N₂ and CO below 5%.

“Tail gas” is used for the remaining stream after one or more products has been obtained from the effluent.

In some embodiments the recycle loop comprises a pre-methanation step in order to reduce the amount of higher hydrocarbons in the recycle loop before methanation. Higher hydrocarbons may be converted to CO and H₂, which CO further may be methanated in the premethanation stage. Especially where the loop comprises an aromatics/gasoline synthesis step the pre-methanation may be advantageous due to the byproducts of the synthesis.

Higher hydrocarbons will tend to form carbon on a regular methanation catalyst, so they may advantageously be pre-reformed/methanated. I.e. for example unsaturated hydrocarbons such as olefins in the effluent/tail gas from the aromatics/gasoline synthesis step can be hydrogenated first, as they may form carbon on the pre-reformer/methanator.

Thus where the present process comprises a step for synthesis of gasoline/aromatics, the effluent/tail gas from the gasoline synthesis may advantageously be processed before being recycled back into the OCM reactor by the following steps:

1) Unsaturated hydrocarbons e.g olefins are processed by hydrogenation to higher alcanes as they may otherwise cause undesired carbon formation. Especially C4+ may be a problem.
2) Higher alkanes (ethane, propane, butane etc) may be reformed/premethanated as they may otherwise form carbon on the methanator in 3).

The higher alkanes may be reformed to CO and H2, which later on can be converted into methane, therefore increasing the methane content to the OCM feed stream. Reforming reactions are however endothermic and will suffer from an energy penalty.

In some applications (such as gasoline/aromatics) it may be beneficial to keep the alkanes such as ethane, propane, butane in the feed. These molecules (higher alanes) will not be steam reformed if the methanator temperature is kept low (e.g. 400° C.)

Carbon formation is much more favoured by alkenes than alkanes. Consequently, the process described in this invention allow efficient and reliable operation on the methanation section, and thus to the entire recycle loop.

3) Methanation of syngas (CO, CO₂ and H₂) to methane for recycle/re-processing in OCM reactor,
4) Hydrogen may be removed to levels below 1% or below 0.5%, such as 0.1%. This can advantageously be done by methanating with CO₂ surplus in which case CO₂ may be added upstream the methanation step. CO can be removed to levels below 5000 ppm or below 500 or 100 ppm such as 1 ppm.

As discussed above without efficient tail gas treatment, the OCM process for e.g. gasoline/aromatics may be inefficient from both a carbon and energy perspective. Due to low conversion rates and selectivity in OCM, a highly efficient recycle process as the present will greatly enhance the overall efficiency. I.e. as the once-through conversion in OCM is often not much higher than 10%, and rarely as high as 25%, the hereby suggested improvements in recycle efficiency have a surprisingly large impact on overall OCM system efficiency.

By the present process and plant the recycle stream preferably comprises more than 70% CH₄, more than 90% CH₄, such as 90-99% at the recycle mixing step.

The recycle stream may preferably be treated in the loop in order to comprise H₂ and CO at a concentration below 5%, below 1%, preferably below 0.5% at the recycle addition stage. The CO concentration may advantageously be even lower such as in the ppm level, for example below 5 ppm, as CO is problematic in relation to the OCM process.

Depending on the products withdrawn and/or produced from the OCM effluent the product(s) may be extracted by separation by condensation, distillation, PSA (pressure swing adsorption), N₂ wash or other separation technologies.

The higher hydrocarbons (HHC) reforming+CO methanation step (pre-methanation) may be carried out over a Ni based cat such as a high temperature methanation catalyst such as Topsøes MCR-8 or AR-401 catalyst. Pressure can be 1-80 barg, such as 10-40 bar range. Temperature 150-500° C. such as e.g. 220-450° C. or 180-350° C. For reaction at temperatures below 400° C. no reforming takes place whereas reforming of HHC may take place above 400° C.

The methanation step may e.g. be carried out over a Ni based catalyst. Pressure can be 1-80 barg, such as 10-40 bar range. Temperature 150-700, such as 180-400° C., or 200-450° C. or 220-450° C. Both inlet and reaction/outlet temperature may be within the given methanation temperature ranges. For example the inlet temperature may be 200° C. and the outlet temp may be 500° C. or inlet 250 and outlet 400° C. The temperature increase may depend on the conditions such as how much CO is converted.

The hydrogenation step may be carried out over e.g. Ni or Cu-based catalyst or e.g. Co—Mo based cat. Pressure can be in the 1-80 bar range, such as 10-40 bar range or 5-20. Temperature 150-450, such as e.g. 250-450° C. or 180-400° C. The temperature increase in a hydrogenation step may be small e.g. inlet 200° C. and outlet 210° C.

Thus according to the present process and plant through a series of processes not previously combined for OCM, it is possible to increase energy-, carbon- and feed-efficiency of OCM processes through an almost complete conversion of tailgas into a very high CH₄-content stream with a balanced and preferably very low level of H₂ and CO. Also, through flexible addition of CO₂ to compensate for variations in feedgas composition, securing constant high efficiency (minimum O₂-use) in the OCM process and protect the catalyst against deactivation by carbon formation.

The plant in which the present process is carried out comprises reactors, heaters, coolers, compressors, separators and/or heat exchangers etc as understood from the above description of the process.

In several advantageous embodiments hydrogenation, pre-methanation and/or methanation of tail gas may be carried out in a single boiling water reactor as the preferred temperature conditions for the three stages are similar e.g. around 250° C.+/−15° C. A setup with a boiling water reactor for hydrogenation, pre-methanation and/or methanation may be advantageous in relation to an aromatics/raw gasoline production especially for treating the tailgas for recycle to an OCM process due to the tailgas composition and the requirements to the OCM feed.

In the following the process and plant is further described with reference to the accompanying drawings. The drawings are exemplary and are not to be construed as limiting to the present process and plant.

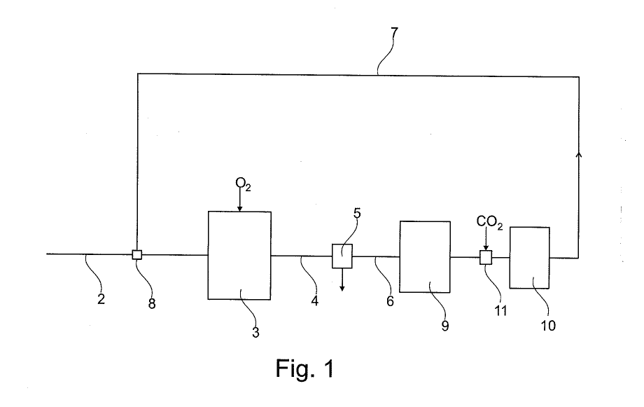

FIG. 1 shows a schematic flow diagram according to some embodiments of the present process and plant 1.

A feed stream 2 comprising CH₄ is provided and enters an OCM conversion stage 3 in which an effluent composed of a multitude of components including but not limited to ethylene, CO₂ and water is produced from the feed and a O₂ stream. An OCM conversion effluent 4 is withdrawn from the OCM conversion step. From this OCM conversion effluent stream Ethylene, water and/or CO₂ can be obtained at one, two or more product retrieval points 5. The remaining effluent 6 is recycled in recycle loop 7 to a recycle mixing point 8 where the recycle is mixed with the feed stream 2.

In the recycle loop the remaining effluent is first converted in a hydrogenation stage 9 and subsequently in a methanation stage 10 in order to remove olefins and obtain a higher CH₄ concentration. Between the hydrogenation stage and methanation stage CO₂ can be added as shown by CO₂ mixing point 11. CO₂ can also be added in the methanation unit between methanation reactors.

It is understood that the process and plant further may comprise stages including compressors, temperature control means such as feed/effluent or steam heat exchangers, electrical heaters, condensers etc.

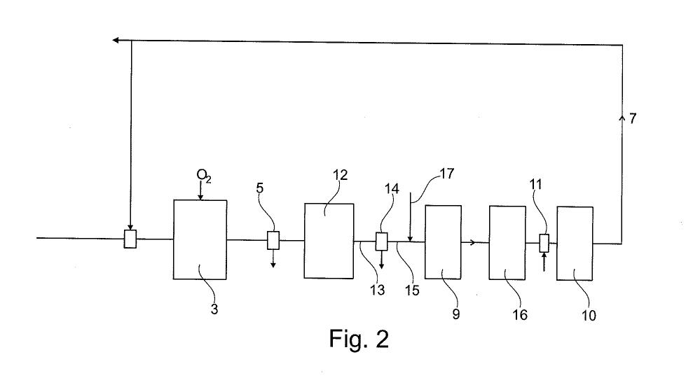

FIG. 2 shows a schematic view of other embodiments of the present process and plant. The basic process and plant parts are known from FIG. 1 and for like parts like numbers are used. The plant and process illustrated in FIG. 2 further comprises a gasoline/aromatics conversion stage 12 in which at least part of the OCM effluent is converted in a gasoline or aromatics synthesis. Raw gasoline and/or aromatics are removed from the stream 13 in a product removal stage 14. The remaining OCM effluent 15 now tail gas from the gasoline and/or aromatics synthesis stage is passed through a hydrogenation stage 9 a pre-methanation stage 16 and finally a methanation stage. Steam may be added if needed e.g. as indicated here 17 upstream the hydrogenation stage.

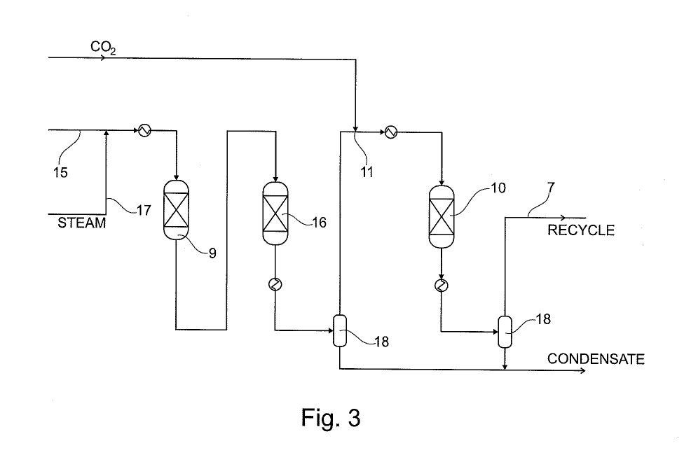

FIG. 3 shows an example of a more detailed recycle loop layout. The basic components are the same as known from previous figures and the same numbers are used. In FIG. 3 water is removed up- and down stream the methanation stage 10 by knock out drums 18.

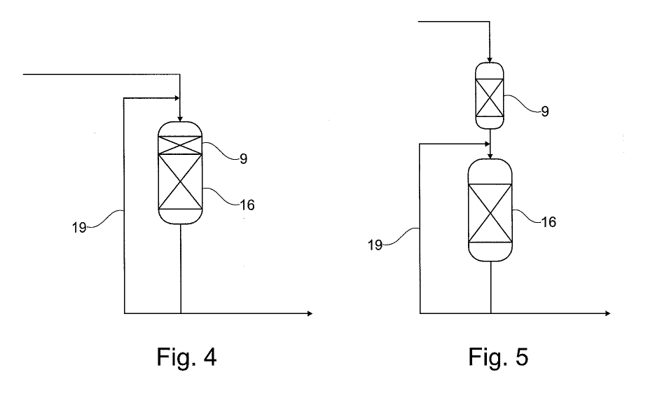

FIG. 4 shows an embodiment with hydrogenation 9 and premethanation 16 of CO, CO₂ and H₂ carried out in two stages in the same reactor. Premethanation may take place together with reforming of alkanes depending on the temperature. For example if T<400° C. in the premethanation step no reforming of higher hydrocarbons will take place. This setup also may include a recycle 19 to utilize water formed in the premethanation step 16. The catalyst in the hydrogenation step may e.g. be a Copper cat such as OS-101. The catalyst in the premethanation/reforming step step may e.g. be a Nickel cat such as AR-401. A methanation step may or may not be arranged downstream the premethanation step if further CO needs to be converted.

FIG. 5 shows a setup similar to that of FIG. 4 but where hydrogenation 9 and premethanation (reforming and/or methanation) 16 are carried out in two separate reactors.

1. A process for production of hydrocarbons, the process comprising the steps of:
converting at least a feed stream comprising CH₄ and O₂ feed into a conversion effluent by oxidative coupling of methane (OCM);
withdrawing one or more product streams from the conversion effluent by at least one product retrieval stage;
recycling at least a portion of the remaining conversion effluent as a recycle stream in a recycle loop, said recycle loop comprising a hydrogenation stage and a methanation stage; and
adding at least part of the recycle stream to the feed stream.

2. Process according to claim 1 wherein the recycle loop further comprises a CO₂ addition stage.

3. Process according to claim 1, wherein CO₂ is added upstream the methanation step at the CO₂ addition stage and/or directly into the methanation unit.

4. Process according to claim 1, wherein H₂O is at least partially removed upstream and/or downstream one or more of the product retrieval steps.

5. Process according to claim 1, wherein the recycle loop comprises a pre-methanation step.

6. Process according to claim 1, wherein CO, CO2 is pre-methanated and/or higher alkanes are reformed in the pre-methanation step.

7. Process according to claim 1, wherein the pre-methanation step is carried out at T<400° C. or >400° C.

8. Process according to claim 1, wherein at least part of the effluent from the premethanation is recycled to upstream the premethanation step, such as upstream hydrogenation or between hydrogenation and premethanation.

9. Process according to claim 1, wherein the product obtained at the one or more product steps are ethylene, CO₂, aromatics and/or raw gasoline.

10. Process according to claim 1, wherein the recycle stream comprises mainly CH₄ at the recycle mixing step preferably where the recycle stream comprises 90-99%, such as above 95% CH₄.

11. Process according to claim 1, wherein the recycle stream comprises H₂ and/or CO at a concentration below 5%, below 1%, below 0.5%, such as below 10 ppm at the recycle mixing point.

12. Process according to claim 1, wherein the one or more products is obtained by separation from the effluent through pressure swing adsorption, condensation, N₂ wash and/or distillation or other separation technologies.

13. Process according to claim 1, wherein the pre-methanation step is carried out over a nickel-based catalyst at a pressure between 0.1 and 80 bars, such as 10-40 bar.

14. Process according to claim 1, wherein the methanation step is carried out over a nickel-based catalyst at a pressure between 0.1 and 80 bars, such as 10-40 bar.

15. Process according to claim 1, wherein the methanation step is carried out at T<400° C. or T>400° C., depending on if the unsaturated hydrogenation is desired or not.

16. Process according to claim 1, wherein the hydrogenation, pre-methanation and/or methanation is carried out in a boiling water reactor preferably in a single boiling water reactor.

17. Plant for production of hydrocarbons, the plant comprising:
an OCM stage,
one or more product retrieval stages, and
a recycle loop comprising at least a hydrogenation stage and a methanation stage.

18. Plant according to claim 17 comprising a hydrocarbon to aromatics conversion stage.

19. Plant according to claim 17, wherein the recycle loop further comprises a pre-methanation stage.

20. Plant according to claim 17, wherein the methanation stage comprises two or more serially arranged methanation steps.

21. Plant according to claim 17, wherein the methanation stage comprises a H₂O removal stage and/or one or more methanation recycles.

22. Plant according to claim 17, comprising a boiling water reactor comprising the hydrogenation, pre-methanation and/or methanation stage.
